(19) United States
(12) Patent Application Publication
(10) Pub. No.: US 2014/0220163 A1
(43) Pub. Date: Aug. 7, 2014

(54) POWDER MIXTURE COMPOSITION OF NATURAL MATERIALS FOR CONTROLLING DEFICIENCIES IN POLYPEPTIDE ALPHA AND BETA HEMOGLOBIN CHAINS

(71) Applicant: Hamzeh Soleimani Babadi, Masjed Soleyman (IR)

(72) Inventor: Hamzeh Soleimani Babadi, Masjed Soleyman (IR)

(21) Appl. No.: 14/172,548

(22) Filed: Feb. 4, 2014

(51) Int. Cl. A61K 36/899; A61K 36/23; A61K 36/534; A61K 36/21; A61K 36/31; A61K 36/482; A61K 36/738; A61K 36/63; A61K 36/282; A61K 36/55; A61K 36/185; A61K 36/61; A61K 36/88; A61K 36/86; A61K 36/87; A61K 36/752; A61K 36/53; A61K 36/28; A61K 36/8967; A61K 36/48; A61K 36/29; A61K 36/84; A61K 36/235; A23L 1/29; A61K 36/484

(52) U.S. Cl. CPC A61K 36/899; A61K 36/484; A61K 36/23; A61K 36/534; A61K 36/21; A61K 36/31; A61K 36/482; A61K 36/738; A61K 36/63; A61K 36/282; A61K 36/55; A61K 36/185; A61K 36/61; A61K 36/88; A61K 36/86; A61K 36/87; A61K 36/752; A61K 36/53; A61K 36/28; A61K 36/8967; A61K 36/48; A61K 36/29; A61K 36/84; A61K 36/235; A23L 1/296
USPC 424/733

(57) ABSTRACT

The embodiments herein provide a powdered composition of natural substances for controlling deficiencies in polypeptide alpha and beta hemoglobin chains and a diet plan for treating thalassemia patient. The composition comprises passargad powder, pars powder, purgative material, fumigation material, snuffing material, vinegar syrup and parsomash powder. The passargad powder is a stomach tonic and acts as anti-parasitic, anti-diarrheal, anti-dysentery, anti-bloating and anti-heartburn agents. The pars powder comprises sprouted grains for providing nutrition to cells. The purgative material is body purifier or detoxification agent and helps in motion and bowl movement. The vapor/fumigation material softens and reduces stickiness of sputum and removes sputum. The snuff helps in sneezing and removal of sputum. The vinegar syrup breaks down fat and removes toxins. The parsomash is an detoxification agent. The composition and dietary plan eliminates the need of bone marrow transplant, blood transfusion and defroxamin in thalassemia patients.

### CROSS REFERENCE TO RELATED APPLICATIONS

The application claims the priority of U.S. provisional patent application with Ser. No. 61/760,219 filed on Feb. 4, 2013 and having the title, “Powder Mixture Based on Natural Substances to Lessen the Abnormal Effects of Polypeptide Alfa and Beta Hemoglobin chain” and the contents of which is incorporated in entirety as reference herein.

### BACKGROUND

1. Technical Field

The embodiments herein generally relate to a field of treatment, prevention, and/or controlling the abnormal effects of metabolic disorder. The embodiments herein particularly relate to a medical therapy for the treatment of abnormal effects of polypeptide alpha and beta hemoglobin chains. The embodiments herein more particularly relate to a powder mixture composition of natural materials to control/treat the abnormal effects of polypeptide alpha and beta hemoglobin chains.

2. Description of the Related Art

Hemoglobin is an iron-containing oxygen transport metalloprotein in the red blood cells of all vertebrates as well as the tissues of some invertebrates. The hemoglobin in the blood carries oxygen from the respiratory organs (lungs or gills) to the rest of the body (i.e. the tissues). The hemoglobin releases the oxygen in tissues to burn nutrients to provide energy and hence power's the functions of the organism by a process called metabolism.

In mammals, the protein hemoglobin makes up about 96% of the dry weight of red blood cells and around 35% of the total content (including water). Hemoglobin has an oxygen binding capacity of 1.34 mL oxygen per gram of hemoglobin, which increases the total blood oxygen capacity by seventy times compared to the dissolved oxygen in blood. The mammalian hemoglobin molecule carries up to four oxygen molecules. The hemoglobin has a quaternary structure, which is a characteristic of many multi-subunit globular proteins. Most of the amino acids in hemoglobin form alpha helices connected by short non-helical segments. The hydrogen bonds stabilize the helical sections inside the hemoglobin protein, causing attractions within the molecule and folding each polypeptide chain into a specific shape. Hemoglobin quaternary structure comes from its four subunits in roughly a tetrahedral arrangement.

In most vertebrates, the hemoglobin molecule is an assembly of four globular protein subunits. Each subunit is composed of a protein chain tightly associated with a non-protein heme group. Each protein chains arranges into a set of alpha helix structural segments connected together in a globin fold arrangement, so called because this arrangement is the same as myoglobin. This folding pattern contains a pocket that strongly binds the heme group.

A heme group consists of an iron (Fe) ion (charged atom) held in a heterocyclic ring known as porphyrin. The porphyrin ring consists of four pyrrole molecules cyclically linked together with the iron ion bound in the center. The iron ion which is the site of the oxygen binding coordinates with the four nitrogen molecules in the center of the ring. The iron is bound strongly (covalently) to the globular protein via the imidazole ring of F8 histidine residue.

In adult humans, the most common hemoglobin type is a tetramer (which contains 4 subunit) called hemoglobin A, consisting of 2α and 2β subunits non-covalently bound each made of 141 and 146 amino acid residues respectively. This is denoted as α₂β₂. The subunits are structurally similar and about the same size. Each subunit has a molecular weight of about 16,000 daltons, for a total molecular weight of the tetramer of about 64,000 daltons.

Thalassemia is an autosomal recessive blood disorder that originated in the Mediterranean region. The thalassemia disorder is caused by weakening and destruction of red blood cells (RBC). Genetically the thalassemia disorder is caused by variant or missing genes that affect how body makes hemoglobin. People with thalassemia make less hemoglobin and fewer circulating red blood cells than normal, which results in mild or severe anemia.

Thalassemia can cause significant complications, including iron overload, bone deformities, cardiovascular illness, slowed growth rate, enlarged spleen and increased risk of infection.

Hemoglobin which contains two pairs of polypeptide chain is the main part of red blood cell. If one of these chains is defected or is not formed at all, leading to thalassemia. The more deficiencies in hemoglobin chains, the more severe is the thalassemia. Thalassemia is prevalent all around the world is caused by blood disorder or genes destruction. It is ranked as one of the costly diseases in the world. The annual cost of treatment for every major thalassemic patient is $20,000 to $100,000 for each patient for 15 years of living. 7% (more than 200 million persons) of the world population is affected all around the world. Control, treatment, and care for thalassemic patients have imposed a great financial and mental pressure on families and the societies.

Regarding thalassemia, many different treatments and medicines have been proposed so far, but due to malfunctioning, incompleteness, and side effects they were stopped at laboratory level and did not go through mass production. Among these is thalassemia medicine that Kumar et al. patented it in U.S. Patent Office in 1996 under the U.S. Pat. No. 5,665,392. The medicine was a mixture of anemonin pretensis and quinine along with aqueous solvent and alcoholic solvent that could reduce the ferritin level in some thalassemic patients and was used as a treatment.

Another research was done by Niharra and colleagues in 1997 and patented in U.S. Patent Office under the U.S. Pat. No. 5,693,671. It revealed that eating L-glutamine and its derivatives could cause reduction in abnormal blood cells, pain and thalassemic symptoms. However, this research was stopped in pilot level due to some problems. Another U.S. Pat. No. 6,372,213 was done by Lee Hum et al. in 2002 and patented in U.S. Patent Office showed that using 1 to 50 doses of micrograms over kilograms over hour of activated protein c along with NaCl injected intravascularly could remove the symptoms and treat thalassemia.

Another European patent EP1952812A1 discloses mixed derivatives of furocoumarin such as acyl furocoumarin, alkyl furocoumarin and methoxyfurocoumarin daily between 8 to 12 times under UVA rays to activate the medicine that could remove the symptoms and improve the sicked cells function by reconstruction of blood factors.

In another research done in 2006 and published in 37th volume of “Blood cells, Molecules and Diseases” magazine by Nishono et al., it is said that using 2.5±0.2 g/dL which is a carrier recombinant of gastro virus for 15 to 30 days leads to 33% increase in hemoglobin and treats β thalassemia. Nowadays, most of the treatment methods are based on deferiprone which was introduced in 1985 for the first time and its effectiveness was proved after a lot of researches. The functionality of this medicine is based on chelating the iron in Erythrocyte membrane. Results of this research were published in “Birith Journal of Hematology” Nom. 75 in 1990.

Also, in another article published in “British Journal of hematology” No. 90 by Rachmiltz et al., the positive effects of Erythropoietin with dose of 500 to 950 units over kilogram on increasing the total and F hemoglobin in patients who took 3 times a day of this medicine were noticed. Although there have been a lot of researches and patents regarding thalassemia, none of them was successful as a prevalent medicine for treatment.

Minor thalassemia is neither preventable nor treatable. For major thalassemia, long term blood transfusion accompanied with deferoxamine and bone marrow transplants are the currently available therapies.

There exist several problems with blood transfusion. The blood bags need to be imported and are expensive. Each blood bag cost is 3,000,000-4,000,000 Iranian Rials. Further each thalassemic patient needs twice a month blood transfusion (1 bag per time). Further the patient must spend 6 hours twice a month. The patients can get an overload of iron after each transfusion that can cause other disease or death which frequently happens. The patients get stressed due to hospital visits and spending 6 hours for blood transfusion.

The “deferoxamine” also has problems such as its rarity in the market (the Iranian version does not match the quality of its foreign version). The deferoxamine must be administered per day throughout the lifetime of patient. One vial of deferoxamine costs 2,00,000 Iranian Rials. The deferoxamine is mainly used for adults and pediatric version is very rare. The deferoxamine has several side effects such as heart failure, kidney failure, heart failure.

The bone marrow transplant has many problems such as the bone marrow donor should be patient's family member (like sister or brother). The bone marrow transplant surgery may not be successful. Further, there is 75-85% success in this bone marrow transplant surgery. It is a fact that bone marrow transplant is the most expensive surgery in the world. It costs more than 1,000,000,000 Iranian Rials (40,000,000-50,000,000 Iranian Rials in public hospitals). In Europe 10,000,000,000 Iranian Rials (around $450,000) and in USA 20,000,000,000 Iranian Rials (around $850,000).

Further, the patients should be kept in hospital for 10-90 days after surgery and the patients must be kept under supervision for 2 years. After surgery, the patients suffer from minor type of thalassemia. Hence the treatment is just a cover for disease. It doesn't cure but increases the side effects. One fact which cannot be ignored is that the bone marrow transplant is the only treatment available for thalassemia patients.

Hence there is a need for powdered composition of natural substances and dietary plan to reduce the thalassemia problems and to provide a treatment therapy for thalassemia at an economical cost. Also there is a need for a powdered composition of natural substances to eliminate a need for blood transfusion and to reduce the rate of child birth affected by thalassemia. Further there is a need for a powdered mixture composition of natural substances to lessen the abnormal effects of polypeptide α and β hemoglobin chain.

The above mentioned shortcomings, disadvantages and problems are addressed herein and which will be understood by reading and studying the following specification.

### OBJECTIVES OF THE EMBODIMENTS

The primary object of the embodiments herein is to provide a simple and cost effective therapy involving powdered compositions of natural substances and dietary plan for the treatment of thalassemia.

Another object of the embodiments herein is to provide a powdered composition of natural substances for controlling the thalassemia symptoms and problems associated with thalassemia.

Yet another object of the embodiments herein is to provide a powdered composition of natural substances for eliminating the need for blood transfusion.

Yet another object of the embodiments herein is to provide a powdered composition of natural substances for eliminating the need for bone marrow transplant.

Yet another object of the embodiments herein is to provide a powdered composition of natural substances for eliminating the need for the administration of defroxamin.

Yet another object of the embodiments herein is to provide a therapy involving powdered compositions of natural substances and dietary plan for reducing the cost of medicine, cost of treatment and patient care.

Yet another object of the embodiment herein is to provide a therapy involving powdered compositions of natural substances and dietary plan for reducing the number of patients referred to clinics and hospitals for blood transfusion.

Yet another objective of the embodiment herein is to provide a powdered composition of natural substances for avoiding the side effects of blood transfusion and blood overload.

Yet another objective of the embodiment herein is to provide a powdered composition of natural substances for eliminating the side effects of the bone marrow transplant.

Yet another objective of the embodiments herein is to provide a powdered compositions of natural substances for eliminating the side effects of defroxamin.

These and other objects and advantages of the embodiments herein will become readily apparent from the following detailed description taken in conjunction with the accompanying drawings.

### SUMMARY

The various embodiments herein provide a new therapy comprising powdered compositions of natural substances and dietary plan for the treatment of thalassemia.

According to one embodiment herein, the powdered mixture composition for controlling deficiencies in polypeptide alpha and beta hemoglobin chains for treating thalassemia patient comprising a passargad powder, a pars powder, a purgative material, a parsomash powder, a vinegar syrup, and a snuffing material,

According to one embodiment herein, the passargad powder comprises 60 grams of liquorice, 60 grams of caraway, 10 grams of anise, 10 grams of mint, and 10 grams of sweet anise.

According to one embodiment herein, the pars powder is a mixture of sprouted grains and wherein the sprouted grains comprise a wheat germ, an oat, an alfalfa, a rice, a lentil, a bean, a vetch, a black grain, a spinach bud, a cress bud, a pea and a bean

According to one embodiment herein, the purgative material comprises a powdered mixture of 50 grams of senna leaves, 50 grams of rose, 50 grams of manna, 25 grams of absinthium, 25 grams of flax and 25 grams of marshmallow.

According to one embodiment herein, the vapour/fumigation material comprises a powdered mixture of 350 grams of coriander, 25 grams of thyme, 25 grams of marjoram, 25 grams of Ziziphora clinopodiodes, 25 grams of pennyroyal, 25 grams of mint, and 25 grams of eucalyptus.

According to one embodiment herein, the snuffing material is powdered mixture of two units of a white hellebore, one unit of coriander and 0.5 units of violet flower.

According to one embodiment herein, the vinegar syrup is a mixture of vinegar and grape syrup at a ratio of 10% and 90%.

According to one embodiment herein, the parsomash a powdered mixture comprising 20 grams of dried limes, 35 grams of mountain tea leaves, 20 grams of oxtongue, 20 grams of lilies, 15 grams of marshmallow, 15 grams of round leaved mallow, 20 grams of dittany, 20 grams of thyme, 30 grams of orange flower, 20 grams of red tea, 20 grams of wormwood, 30 grams of lettuce seed, 20 grams of coriander seed, 20 grams of purslane seed, 15 gram of dill seed, 15 grams of basil seed, 15 grams of succor seed, 25 grams of barberry, 15 grams of valerian, 30 grams of teazle, 15 grams of lavender, 15 grams of lemon balm, 15 grams of mint leaves, 15 grams of chamomile, 15 grams of maidenhair, 15 grams of violets, and a 15 gram fennel.

According to one embodiment herein, the passargad powder is provided in form of powder directly or as a tablet. The passargad powder is directly administered to the thalassemia patient. The passargad powder is given to thalassemia patients 3 times a day before each meal.

According to one embodiment herein, the pars powder is taken one spoonful three times a day by the thalassemia patients.

According to one embodiment herein, the vinegar syrup is taken 2-3 times a day in a quantity of 3-5 spoons.

According to one embodiment herein, the parsomash is taken by thalassemia patients as tea and wherein the parsomash is a detoxifying agent.

According to one embodiment herein, the diet plan for thalassemia patient comprises a diet plan for anemia, a diet plan for softening phlegm, and a diet plan for excreting the phlegm.

According to one embodiment herein, the diet plan for anemia comprises a currant, a fig, a prune, a jujube, a white basil seeds (dragon seeds), a sap taken from sheep backbone, a vinegar syrup, a passargad powder and a pars powder.

According to one embodiment herein, the diet plan for softening the phlegm comprises a flexweld and a manna, a plantain along with fumigation/vaporization.

According to one embodiment herein, the diet plan for excreting the phlegm comprises a purgative and a snuffing material.

According to one embodiment herein, the diet plan comprises a breakfast, a lunch and a dinner. The breakfast comprises a drink comprising manna and flixweld, a drink comprising white basil seeds and dragon head dissolved in rosewater and Bidmeshk, pars powder, grape sap and fruits comprising plantain, prune, fig and jujube.

According to one embodiment herein, the lunch comprises a teaspoon or one or two tablets of passargad powder before lunch, meals comprising a pottage with beans, a green pea stew, sheep's rumen, abdomen and lung, cauliflower soup, cooked rice with coriander, sap taken from a sheep backbone, a grape sap, and a pars powder, and a purgative material after lunch.

According to one embodiment herein, the dinner comprises a teaspoon or one or two tablets of passargad powder before dinner, a meals comprising cooked rice with coriander or a boiled zucchini and fowl meat or a sheep's rumen, a sap taken from sheep backbone, grape sap, and pars powder.

According to one embodiment herein, the snuffing, fumigation and oiling the nostrils are carried out before sleep.

According to one embodiment herein, the powdered composition of natural substances for the treatment of thalassemia comprises passargad powder, pars powder, purgative material, fumigation material, snuffing material and parsomash.

According to one embodiment herein, the passargad powder comprises liquorices, a caraway, an anise, a mint, a sweet anise. The powder of all the components is mixed with honey and water. Further the mixture is shaped into tablets and dried. These tablets must be taken 3 times a day before meal.

According to one embodiment herein, the pars powder is a mixture of wheat germ, grains, oats, alfalfa, rice, lentil, been, vetch is added to make a powder. The powder must be taken one spoon full 3 times a day.

According to one embodiment herein, the purgative is a powder mix of senna leaves, rose, manna, absinthium, flax, marshmallow boiled in 2 glass of water. The medicine should be taken in a way that causes four times of purge a day that continues for 20 days.

According to one embodiment herein, the fumigation material comprises coriander, thyme, marjoram, Ziziphora clinopodiodes, pennyroyal, mint, eucalyptus. The mixture is boiled in pot and the steam is inhaled for 10 minutes every night before going to bed.

According to one embodiment herein the snuffing material comprises, the powder of two units of white hellebore, one unit of coriander, 0.5 unit of violet flower. The powder must be inhaled. The inhalation will lead to sneezing. After clearing the nose congestion or cleaning the nose, violet flower oil or almond oil is dropped into nostrils.

According to one embodiment herein, the mixture of vinegar and syrup comprise vinegar 10% and grape syrup 90%. The mixture is taken 2-3 times in a quantity of 3-5 spoons.

According to one embodiment herein, the diet plan for treating anemia in thalassemia patient comprises currant, fig, prune, jujube, white basil seeds (dragon head), sap from sheep backbone, vinegar and grape syrup, passargad powder and pars powder.

According to one embodiment herein, the diet plan for softening the phlegm in thalassemia patient comprises flixweld and manna, plantain. For softening the phlegm along with dietary regimen fumigation is necessary.

According to one embodiment herein, the diet plan for excreting the phlegm in thalassemia patient comprises purgative, snuff and violet flower oil and a combination of lotus, myrtle, henna, vinegar.

According to one embodiment herein, the boiled food is fed for at least two months for thalassemia patient. After two months, olive oil, sesame oil or ghee is added. Further three glasses of fresh fruit juice are given to the thalassemia patient. The thalassemia patients must avoid egg, fatty food, fried food, solid or liquid oil, sheep's head and leg, fizzy beverage, sausage, chips, snacks, ice cream, canned food, cold water, diaries (specially pasteurized milk, homogenized milk and yoghurt), pickle, cream, rinsed rice, water or any other fluid half an hour before and after meal.

According to one embodiment herein, addition of chilly and garlic is avoided in the diet of thalassemia patient, when the thalassemia patient has skin or inflammatory disease like hives, eczema etc

According to one embodiment herein, the patient is defecated four times in a day. The patient is administered with the purgative materials to have a diarrheal effect. When the patient does not have the diarrheal symptom even after the administration of purgative material, then the amount of purgative material administered is to be increased from one cup to three cups. Further the thalassemia patients are fed with fig and prune to have a diarrheal effect. The children are administered with half a cup of purgative material. The diarrhea is controlled with honey and dill. In some cases the symptoms of nausea and stomach cramps are found after the administration of purgative material. In order to prevent stomach cramps, the medicine is fed as a warm drink for a period of half an hour.

According to one embodiment herein, a method for obtaining the sap from sheep backbone comprises boiling 1.5 Kg of sheep backbone with salt, onion and turmeric in a pot. The soft bones are pressed to remove the water out of it. The riffraff is separated and cooked in the sap to prepare a food for thalassemia patient. Also fresh spinach, coriander and dill leaves are added in the food. The sheep backbone sap is used once or twice a week or 3 cups are used every day.

According to one embodiment herein, the diabetic thalassemia patient must avoid sweets in diet and in powdered composition of natural substances. The thalassemia patients with cold must avoid grape sap. The patients can use brown sugar.

According to one embodiment herein, parsomash is a kind of herbal tea made up of plant leaves, flowers and seeds. The parsomash herbal tea is prepared by mixing 3 spoonfuls of the parsomash herbal composition with 3 glasses of boiling water and brewing it for half an hour. One glass of the parsomash herbal tea composition is taken 3 times daily.

According to one embodiment herein, the following steps are followed for providing a bath during the treatment of thalassemia patient. The powders of lotus, myrtle and henna are mixed in the ratio of 150 gms, 500 gms, and 350 gms in one glass of a vinegar or a lemon juice and a little water to prepare a solution. The prepared solution is applied on all parts such as body face and head of the thalassemia patients and massaged for 10 minutes. After rubbing the skin of the thalassemia patients for half an hour, the skin is washed with water and without using any shampoo or soap until the end of the treatment period.

The diet plan for breakfast of the thalassemia patient is as follows. The breakfast comprises a composition to treat anemia. The composition is prepared by dissolving 3 spoons full of manna into 1.5 glass of boiling water and adding 3 spoonful of cleaned flixweld. The manna and flixweld are given as drinks in the breakfast. Equal amount or 1 teaspoon each of white basil seeds and dragon head are mixed in 1.2 cups of rosewater and 1.2 cups of Bidmeshk. The mixture is given as drink after an elapse of 1.5 hours since taking breakfast. The pars powder, grape sap and currant are fed after half an hour. The plantain, prune, fig and jujube are fed after an hour.

The diet plan for the lunch for the thalassemia patient is as follows. A teaspoon or one or two tablets of passargad powder is given before lunch. The food in the lunch comprises a pottage with beans, a green pea stew, sheep's rumen, abdomen and lung, cauliflower soup, cooked rice with coriander; the sap taken from a sheep backbone, a grape sap and a pars powder. A purgative material is given to the patient after lunch

The diet plan for the dinner of the thalassemia patient is as follows. The diet plan for the dinner comprises a medicine or composition for treating the anemia. A teaspoon or one or two tablets of passargad powder is given to the patient before food in the dinner. The food in the dinner comprises cooked rice with coriander or a boiled zucchini and fowl meat or a sheep's rumen, a sap taken from sheep backbone, grape sap and pars powder. A snuffing, fumigation and oiling of the nostrils are done before sleep.

According to one embodiment herein, the powdered composition of natural substances/materials and dietary plan for the treatment of thalassemia, has reduced the need for blood transfusion. By taking the powdered composition of natural substances and following the dietary plan, the patients have eliminated the need for a frequent visit to the clinics and hospitals. Further the thalassemia patients are advised not to take medication to remove an overload of iron. The powdered composition and dietary plan for the treatment of thalassemia reduces/eliminates the side effects due to an overdose of iron Also the blood chemistry is maintained at normal level. The powdered compositions and diet plan improves and removes the deficiency of polypeptide alfa and beta. Further the improper alfa and beta hemoglobin chains are normalized, thereby maintaining the blood chemistry and blood factors at normal level.

These and other aspects of the embodiments herein will be better appreciated and understood when considered in conjunction with the following description and the accompanying drawings. It should be understood, however, that the following descriptions, while indicating the preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, as reference is made to the accompanying drawings that form a part hereof, and in which the specific embodiments that may be practiced is shown by way of illustration. These embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a powdered composition of natural substances and a dietary plan for the treatment of abnormal effects of polypeptide alpha and beta hemoglobin chains. The embodiments herein also relate to a powdered mixture of natural materials to control/treat the abnormal effects of polypeptide alpha and beta hemoglobin chains in thalassemia patients. Currently there are no medicines that keep the blood factors at a normal level in thalassemia patients. Further, the treatments available for the thalassemia patients are a blood transfusion with doxorubicin and a bone marrow transplant. The therapies have several side effects. The powdered composition of natural materials and dietary plan eliminates the need for a surgery.

The various embodiments herein provide a powdered composition of natural materials or native substances and a dietary plan for the treatment of thalassemia. The powder composition of natural substances for the treatment of thalassemia comprises passargad powder, pars powder, purgative material, vapor/fumigation material, snuffing material and vinegar syrup.

According to one embodiment herein, the passargad powder comprises 60 grams of liquorice, 60 grams of caraway, 10 grams of anise, 10 grams of mint, and 10 grams of sweet anise. The powder of all the above-mentioned materials is mixed with honey and water to obtain a mixture. Further the mixture is transformed into the tablet shape and dried. These tablets are taken 3 times a day before meal.

According to one embodiment herein, a simple method for the synthesis of the passargad powder comprises crushing the ingredients to obtain a powder of mixture. The ingredients comprises 60 grams of liquorice, 60 grams of caraway, 10 grams of anise, 10 grams of mint, 10 grams of sweet anise. The powder mixture is mixed with sugar or cubic sugar. One tablespoon of the powder mixture is taken by thalassemia patient before every meal with a little water. The powder is also be mixed in water and taken.

The “passargad” is an Iranian name that refers to the above mentioned powder mixture. The passargad powder mixture acts or functions as a stomach tonic, an anti-parasitic agent, an anti-diarrheal agent, an anti-dysentery agent, an anti-bloating agent and an anti-heartburn agent. The passargad powder mixture also eliminates bellyache and nausea. Further the passargad powder mixture functions as a liver booster and kills Helicobacter bacterium. The passargad powder composition strengthens stomach for better and easier digestion of food. Also passargad powder composition prevents side effects like nausea, diarrhea and bloating. The passargad powder composition also helps the people with ulcers and stomach upset/indigestion. The passargad powder composition also facilitates digestion and absorption of nutrients.

According to one embodiment herein, the pars powder is a mixture of sprouted grains and comprises a wheat germ, an oat, an alfalfa, a rice, a lentil, a been, and a vetch powder. The powder is prepared by crushing 2 units each of the wheat germ, the grain, the oats, the alfalfa, the rice and the lentil and 1 unit each of a sprouted black grain, a spinach bud, a cress bus, a pea, a bean (such as mung bean etc), and a vetch. One spoon full of the pars powder is taken 3 times a day by thalassemia patient.

The “pars” is an Iranian name that refers to the mixture mentioned above. The sprouted grain, wheat and beans disintegrate the fat and starch available in them into smaller units. The disintegrated molecules are better absorbed for their nutrients such as vitamins, minerals and protein. The absorption of molecules is increased by 2-3 times more than that of the non-sprouted grain, wheat and beans. Also the carbohydrate and calorie in the sprouted grain, wheat and beans are decreased by two times. Further the sprouts are a rich source of amino acid, natural sugars and nutrients that are readily absorbed by the body. Eating the sprouts causes the alkaline and acidic condition that leads to better function of the kidneys, the circulatory system and the respiratory system.

The wheat germ consists of vitamin E and superoxide dismutase. Vitamin E and superoxide dismutase are essential antioxidants to protect the cells that are structurally changed. The sprouted lentil works as a blood purifier, kidney cleaner and adrenal gland stimulator. The sprouted mung bean detoxifies body, acts as diuretic and is a rich source of folic acid. The sprouted alfalfa reduces the vascular deposition. The sprouted alfalfa contains calcium and iron which is two times more than that present in spinach.

The main role of the pars powder for the treatment of Thalassemia patient is to provide the cells with nutrients for better metabolic activity. Further the pars powder provides the cells with nutrients for better activity and provides iron. The pars powder also strengthens heart, different organs and helps in dispelling the sputum.

According to one embodiment herein, the purgative material is a powder mixture of 50 grams of senna leaves, 50 grams of rose, 50 grams of manna, 25 grams of absinthium, 25 grams of flax, 25 grams of marshmallow. One spoonful of the powder is boiled in 2 glasses of water to obtain a concoction. The boiled concoction is filtered and sweetened. The concoction is taken after lunch. The medicine is taken in a way that causes four times of purging a day and the purging style is continued for 20 days. If the patient does not have a proper purging it is recommended to increase the amount of intake of purging material and vice versa.

The purgative agent is a strong native or natural composition suitable for use when the patients need a stomach wash or cleaning. The purgative natural composition lubricates the gut. Further purgative natural composition absorbs and dispels the sputum. The softened sputum is directed towards intestines and the sputum is expelled/discharged from the body through purgative. The purgative composition facilitates removal or discharge of toxins. Further by removing the toxins from the body, the metabolic malfunctioning and the deficiencies of organs are eliminated.

According to one embodiment herein, the fumigation material/vapor composition comprises 350 grams of coriander, 25 grams of thyme, 25 grams of marjoram, 25 grams of Ziziphora clinopodiodes, 25 grams pennyroyal, 25 grams of mint, and 25 grams of eucalyptus. The mixture is boiled in pot and the steam is inhaled for 10 minutes every night before going to bed.

The fumigation material/vapor composition is capable of disinfecting the brain and brain membrane. The sputum is lead downward after the fumigation. The fumigation material/vapor composition clears the nose ducts, throat, larynx, ear and eyes. The purpose of fumigation or vaporization is to soften and reduce the stickiness of the sputum. When the sputum from brain is removed/discharged, the brain function is improved and prevented from sending erroneous messages to the body organs. Further the fumigation material/vapor composition paves the way for sneezing. The sneezing discharges more waste.

According to one embodiment herein, the snuffing material comprises a powder mixture of two units of white hellebore, one unit of coriander and 0.5 unit of violet flower. One teaspoon of the powder mixture is inhaled by thalassemia patient. The inhalation leads to a sneezing of more than 15-20 times. After clearing/cleaning of the nose, violet flower oil or almond oil is dropped into nostrils. The snuffing is done 10 minutes after fumigation. The violet flower belongs to the group of flowering plants category: Phylum-dicotyledonous, Kind-Malphighiales, violaceae. Scientifically the violet flower is called Viola pansy, Viola Teicolor, Viola pansee or wild pansy.

The snuffing material is a combination of aromatic medicine, drug and the snuffling medicine is dropped/packed into nose. Once the sputum is softened, then the sputum is removed/discharged from the body by sneezing. The sneezing is induced by snuffing. The sneezing prevents different kinds of diseases. The sneezing accelerates blood circulation in head and clears the vascular and endobiotic circulation. The sneezing improves the eyesight, removes fatigue and clears the heart, kidneys, septum and larynx from any deposition. The sneezing reduces blood clotting in heart or brain. The toxins combine with sputum and cause disorders. The primary role of sneezing in thalassemia patients is to remove/discharge sputum from the body to prevent disorders.

According to one embodiment herein, the mixture of vinegar and syrup is prepared by mixing vinegar and grape syrup at a ratio of 10% and 90%. The mixture is taken 2-3 times in a quantity of 3-5 spoons. The combination of vinegar and grape syrup is called a “vinegar syrup” which is a traditional Iranian syrup, that acts both as drug and food.

The vinegar syrup has the properties of both vinegar and syrup. The combination negates the harmful effects of vinegar alone. The vinegar syrup helps in better digestion, acts as an appetizer, helps to stay in shape, increases vigilance and reduces anemia. The vinegar syrup cleanses liver, digestive system and spleen. The vinegar syrup facilitates in discharging the sputum. The grape sap acts as energizer, hematopoietic stimulant of liver. Further the vinegar helps in a breaking down of fat and discharges/removes the toxins. In totality, the purpose of using vinegar syrup is to provide energy, blood and to reduce anemia.

The thalassemia patients having vinegar syrup must avoid dairies. The dairies can be taken 3 hours after or before having vinegar syrup. Further vinegar syrup is not advisable to the patients suffering from neurasthenia, ulcer, diabetes and joint pain. The thalassemia patients must avoid industrial vinegar, as the industrial vinegar does not have same properties as natural vinegar. For preparation of the vinegar syrup, the grape vinegar and grape sap are used.

According to one embodiment herein, the diet plan for treating anemia in thalassemia patient involves using currant, fig, prune, jujube, white basil seeds (dragon head), sap from sheep backbone, vinegar and grape syrup, passargad powder and pars powder.

According to one embodiment herein, the diet plan for softening the phlegm in thalassemia patient includes a flixweld, a manna, and the plantain. For softening the phlegm, fumigation is necessarily done along with dietary plan.

According to one embodiment herein, the diet plan for facilitating the excretion of the phlegm in thalassemia patient comprises using the purgative, the snuff and the violet flower oil with a combination of lotus, myrtle, henna, vinegar.

According to one embodiment herein, in the diet plan for thalassemia patient includes the boiled food which is taken for at least two months. After two months, olive oil, sesame oil or ghee are added. Further three glasses of the fresh fruit juice are given to the thalassemia patient.

The thalassemia patients are not given with egg, fatty food, fried food, solid or liquid oil, sheep's head and leg, fizzy beverage, sausage, chips, snacks, ice cream, canned food, cold water, diaries (specially pasteurized milk, homogenized milk and yoghurt), pickle, cream, rinsed rice, water or any other fluid half an hour before and after meal. For the thalassemia patients, any fluid is not given half an hour before and half an hour after meal except vinegar syrup. The fruits are not taken half an hour after or before meal. Smoking, bathing, lying down and walking are avoided half an hour after meal. The thalassemia patients are not fed with canned food in the diet. The diet is kept as simple. The sheep meat, goat meat and olive oil are included in the diet during a recovery process. The thalassemia patients are provided with organic fruits.

According to one embodiment herein, addition of chilly and garlic is avoided in the diet of thalassemia patient, when the thalassemia patient has skin or inflammatory disease like hives, eczema etc.

According to one embodiment herein, the patient is defecated four times in a day. The patient is administered with the purgative materials to have a diarrheal effect. When the patient does not have the diarrheal symptom even after the administration of purgative material, then the amount of purgative material administered is to be increased from one cup to three cups. Further the thalassemia patients are fed with fig and prune to have a diarrheal effect. The children are administered with half a cup of purgative material. The diarrhea is controlled with honey and dill. In some cases the symptoms of nausea and stomach cramps are found after the administration of purgative material. In order to prevent stomach cramps, the medicine is fed as a warm drink for a period of half an hour.

According to one embodiment herein, a method for obtaining the sap from sheep backbone comprises boiling 1.5 Kg of sheep backbone with salt, onion and turmeric in a pot. The soft bones are pressed to remove the water out of it. The riffraff is separated and cooked in the sap to prepare a food for thalassemia patient. Also fresh spinach, coriander and dill leaves are added in the food. The sheep backbone sap is used once or twice a week or 3 cups are used every day.

According to one embodiment herein, the diabetic thalassemia patient must avoid sweets in diet and in powdered composition of natural substances. The thalassemia patients with cold must avoid grape sap. The patients can use brown sugar. The brown sugar is made of sugarcane and it is not purified. The brown color of the sugar is due to the molasses. The brown sugar is semi-refined sugar that still has molasses. The brown sugar contains useful molecules like calcium, magnesium, potassium and sodium.

According to one embodiment herein, parsomash is a kind of herbal tea made up of plant leaves, flowers and seeds. The parsomash herbal tea is prepared by mixing 3 spoonfuls of the parsomash herbal composition with 3 glasses of boiling water and brewing it for half an hour. One glass of the parsomash herbal tea composition is taken 3 times daily. The parsomash acts as detoxifying agent.

The parsomash is a kind of herbal tea composition made up of plants leaves, flower and seeds. The parsomash herbal tea composition is prepared by mixing 20 grams of dried limes, 35 grams of mountain tea leaves, 20 grams of oxtongue, 20 grams of lilies, 15 grams of marshmallow, 15 grams of round leaved mallow, 20 grams of dittany, 20 grams of thyme, 30 grams of orange flower, 20 grams of red tea, 20 grams of wormwood, 30 grams of lettuce seed, 20 grams of coriander seed, 20 grams of purslane seed, 15 grams of dill seed, 15 grams of basil seed, 15 grams of succor seed, 25 grams of barberry, 15 grams of valerian, 30 grams of teazle, 15 grams of lavender, 15 grams of lemon balm, 15 grams of mint leaves and mother-of-thyme, 15 grams of chamomile, 15 grams of maidenhair, 15 grams of violets, and 15 grams of fennel. Three teaspoon full of the parsomash powder composition is mixed in 3 glasses of water. The water with parsomash powder composition is boiled for half an hour. One glass of boiled parsomash herbal tea is taken for three times a day.

According to one embodiment herein, the following steps are followed for providing a bath during the treatment of thalassemia patient. The powders of lotus, myrtle and henna are mixed in the ratio of 150 gms, 500 gms, and 350 gms in one glass of a vinegar or a lemon juice and a little water to prepare a solution. The prepared solution is applied on all parts such as body face and head of the thalassemia patients and massaged for 10 minutes. After rubbing the skin of the thalassemia patients for half an hour, the skin is washed with water and without using any shampoo or soap until the end of the treatment period.

The diet plan for breakfast of the thalassemia patient is as follows. The breakfast comprises a composition to treat anemia. The composition is prepared by dissolving 3 spoons full of manna into 1.5 glass of boiling water and adding 3 spoonful of cleaned flixweld. The manna and flixweld are given as drinks in the breakfast. Equal amount or 1 teaspoon each of white basil seeds and dragon head are mixed in 1.2 cups of rosewater and 1.2 cups of Bidmeshk. The mixture is given as drink after an elapse of 1.5 hours since taking breakfast. The pars powder, grape sap and currant are fed after half an hour. The plantain, prune, fig and jujube are fed after an hour.

The diet plan for the lunch for the thalassemia patient is as follows. A teaspoon or one or two tablets of passargad powder is given before lunch. The food in the lunch comprises a pottage with beans, a green pea stew, sheep's rumen, abdomen and lung, cauliflower soup, cooked rice with coriander; the sap taken from a sheep backbone, a grape sap and a pars powder. A purgative material is given to the patient after lunch

The diet plan for the dinner of the thalassemia patient is as follows. The diet plan for the dinner comprises a medicine or composition for treating the anemia. A teaspoon or one or two tablets of passargad powder is given to the patient before food in the dinner. The food in the dinner comprises cooked rice with coriander or a boiled zucchini and fowl meat or a sheep's rumen, a sap taken from sheep backbone, grape sap and pars powder. A snuffing, fumigation and oiling of the nostrils are done before sleep.

According to one embodiment herein, the powdered composition of natural substances/materials and dietary plan for the treatment of thalassemia, has reduced the need for blood transfusion. By taking the powdered composition of natural substances and following the dietary plan, the patients have eliminated the need for a frequent visit to the clinics and hospitals. Further the thalassemia patients are advised not to take medication to remove an overload of iron. The powdered composition and dietary plan for the treatment of thalassemia reduces/eliminates the side effects due to an overdose of iron Also the blood chemistry is maintained at normal level. The powdered compositions and diet plan improves and removes the deficiency of polypeptide alfa and beta. Further the improper alfa and beta hemoglobin chains are normalized, thereby maintaining the blood chemistry and blood factors at normal level.

The morning diet plan for the thalassemia patient is mentioned below in Table 1:

| | | |
|---|---|---|
| Before | Flixweld and manna | Dissolve 3 spoonfuls of |
| breakfast | | manna into 1.5 glass of |
| | | boiling water(filter it in case |
| | | of need) and pour it hot on 3 |
| | | spoonfuls of cleaned |
| | | flixweldand have it as your |
| | | breakfast(it is better not to eat |
| | | anything until noon) |
| 1.5 hours | White basil seeds - | Mix them equally. Then mix 1 |
| after | dragon head | teaspoon of it with 1.2 cup |
| breakfast | | rosewater and 1.2 cup of |
| | | bidmeshk(add a little sugar in |
| | | case of need) |
| Half an | Passargad powder | 10 min before breakfast, take 1 |
| hour later | Breakfast | or 2 pea size of the passargad |
| | Grape sap | powder and mix it with water |
| | Pars powder | to make dough. Add a little |
| | Currant | honey and roll it like a tablet. |
| | | Then dry it and eat it before |
| | | meal. |
| | | Then breakfast(it is |
| | | recommended not to eat |
| | | anything until noon) |
| | | 3 to 5 spoonfuls of grape sap |
| | | mixed with water and 1 spoon |
| | | of pars powder should be |
| | | taken with your breakfast |
| | | Finally eat 36 to 72 currant |
| | | infused in water from a night |
| | | before |
| 1 hour | fleawort | Mix 1 spoon of fleawort with |
| later | Prune and fig | 1.2 glass mint sweat and 1.2 |
| (at 10:00 a.m.) | Jujube | glass of boiling water and |
| | | drink it |
| | | Eat 5 figs and 10 prunes |
| | | soaked in water from a night |
| | | before |
| | | Also eat 10 jujubes |

The afternoon diet regimen or diet plan for the thalassemia patient is mentioned below in Table 2:

| Time | Consumption | Description |
|---|---|---|
| Before lunch | Passargad powder | Some min. before lunch take |
| | | 1 or 2 pea size of the |
| | | passargad powder and mix it |
| | | with water to make dough. |
| | | Add a little honey and shape |
| | | it like a tablet. Then dry it |
| | | and eat it. |
| Lunch | food | Eat coriander soup every |
| | | other day cooked as |
| | | following: |
| | | Coriander, spinach or chard, |
| | | lentil, a little rice, sheep or |
| | | chicken meat without fat, |
| | | prune |
| | | And in the following days |
| | | choose one of these: |
| | | 1-barly soup with beans: |
| | | Grain boiled with beans, a |
| | | little garlic, onion, salt and |
| | | chili(you can add a little |
| | | olive oil or lemon juice to |
| | | eat) |
| | | 2-split peas soup: |
| | | Boil mint, parsley, coriander, |
| | | carrot, onion, garlic, a little |
| | | grain and lentil, and fatless |
| | | chicken or sheep meat in case |
| | | you want(add turmeric and |
| | | salt as seasoning) |
| | | 3-sheep's rumen, abomasums |
| | | and lung: |
| | | Take their fat away and boil |
| | | them with onion, salt, |
| | | turmeric and garlic(add a |
| | | little lemon juice and chili to |
| | | eat) |
| | | 4-califlower soup: |
| | | Boil cauliflower, coriander, |
| | | parsley, carrot, chicken or |
| | | sheep's meat(fatless), garlic, |
| | | salt, turmeric and onion. (add |
| | | a little lemon juice and chili |
| | | to eat) |
| | | 5-cooked rice with coriander: |
| | | Rice, coriander, sheep's |
| | | meat(fatless), salt, onion, |
| | | turmeric, and a little garlic |
| | | 6-corn soup: |
| | | Mix and boil corn, lentil, |
| | | grain, carrot, coriander, a |
| | | little garlic, onion, salt and |
| | | turmeric |
| With lunch | Sap taken from a sheep's | Drink 1.5 cups of Sap taken |
| | backbone | from a sheep's |
| | | backbone(prepare it as this |
| | | recipe) |
| | | Boil 1.5 kilo of |
| | | sheep'sbackbone(meatless) |
| | | with a little salt, onion and |
| | | turmeric in a pot every week. |
| | | When bones become |
| | | completely soft press them to |
| | | remove the water out of it. |
| | | Then separate the riffraff and |
| | | cook your favorite food with |
| | | its sap. You can also add |
| | | coriander and dill and |
| | | spinach in your food. Make it |
| | | once or twice a week and use |
| | | it in your soup or food or |
| | | drink 3 cups of its sap daily. |
| | Grape sap | With your lunch, mix 3 to 5 |
| | Pars powder | spoonfuls of grape sap with |
| | | water and 1 spoon of pars |
| | | powder and eat it. |
| In the evening | Purgative | Boil 1 spoon of it with 2 |
| | | glasses of water until 1 glass |
| | | is remained. Then, filter and |
| | | sweeten it to drink. This |
| | | medicine should cause mild |
| | | diarrhea. So, take it once or 3 |
| | | times a day. |

The dinner diet regimen or diet plan for the thalassemia patient is mentioned below in Table 3:

| Time | Consumption | Description |
|---|---|---|
| Before | Pasargad powder | 10 min before dinner, take 1 or |
| dinner | | 2 pea size of the pasargad |
| | | powder and mix it with water |
| | | to make dough. Add a little |
| | | honey and shape it like a |
| | | tablet. Then dry it and eat it. |
| Dinner | Food | Choose one of the following: |
| | | 1-cooked rice with coriander |
| | | or dill with boiled chicken or |
| | | sheep meat |
| | | 2-boiled zucchini and the |
| | | meat of any fowl |
| | | 3-sheep's rumen, |
| | | abomasums, and lung(fatless) |
| | | with lemon juice as |
| | | seasoning |
| With | Sap taken from a sheep's | Drink 1.5 cups of Sap taken |
| dinner | backbone | from a sheep's |
| | | backbone(according to |
| | | mentioned recipe) |
| | Grape sap | With your dinner, mix 3 to 5 |
| | Pars powder | spoonfuls of grape sap with |
| | | water and 1 spoon of pars |
| | | powder and eat it. |
| Before | 1-fumigation | Every night before sleep, boil |
| sleep | 2-snuffing | 1.2 cup of fumigation |
| | 3-oil seeping | medicine in water and and |
| | | inhale it for 10 mins. under a |
| | | cover till your face sweats. |
| | | Then, pour a tiny amount of |
| | | snuffing powder in your |
| | | palm and inhale it. Wait till |
| | | the sneezes stop. After that, |
| | | instill a drop of violet oil or |
| | | almond oil in each nostril. |
| | | The next morning inhale |
| | | washy saltwater. |

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. The powdered mixture composition for controlling deficiencies in polypeptide alpha and beta hemoglobin chains for treating thalassemia patient comprising:
a passargad powder, and wherein the passargad powder comprises 60 grams of liquorice, 60 grams of caraway, 10 grams of anise, 10 grams of mint, and 10 grams of sweet anise;
a pars powder, and wherein the pars powder is a mixture of sprouted grains and wherein the sprouted grains comprise a wheat germ, an oat, an alfalfa, a rice, a lentil, a bean, a vetch, a black grain, a spinach bud, a cress bud, a pea and a bean;
a purgative material, and wherein the purgative material comprises a powdered mixture of 50 grams of senna leaves, 50 grams of rose, 50 grams of manna, 25 grams of absinthium, 25 grams of flax and 25 grams of marshmallow;
a vapour/fumigation material, and wherein vapour/fumigation material comprises a powdered mixture of 350 grams of coriander, 25 grams of thyme, 25 grams of marjoram, 25 grams of Ziziphora clinopodiodes, 25 grams of pennyroyal, 25 grams of mint, and 25 grams of eucalyptus;
a snuffing material, and wherein the snuffing material is a powdered mixture of two units of a white hellebore, one unit of coriander and 0.5 units of violet flower;
a vinegar syrup, and wherein the vinegar syrup is a mixture of vinegar and grape syrup at a ratio of 10% and 90%; and
a parsomash powder, and wherein the parsomash a powdered mixture comprising 20 grams of dried limes, 35 grams of mountain tea leaves, 20 grams of oxtongue, 20 grams of lilies, 15 grams of marshmallow, 15 grams of round leaved mallow, 20 grams of dittany, 20 grams of thyme, 30 grams of orange flower, 20 grams of red tea, 20 grams of wormwood, 30 grams of lettuce seed, 20 grams of coriander seed, 20 grams of purslane seed, 15 gram of dill seed, 15 grams of basil seed, 15 grams of succor seed, 25 grams of barberry, 15 grams of valerian, 30 grams of teazle, 15 grams of lavender, 15 grams of lemon balm, 15 grams of mint leaves, 15 grams of chamomile, 15 grams of maidenhair, 15 grams of violets, and a 15 gram fennel.

2. The composition according to claim 1, wherein the passargad powder is provided in form of powder directly or as a tablet.

3. The composition according to claim 2, wherein the passargad powder is directly administered to the thalassemia patient.

4. The composition according to claim 2, wherein the passargad powder is given to thalassemia patients 3 times a day before each meal.

5. The composition according to claim 1, wherein the pars powder is taken one spoonful three times a day by the thalassemia patients.

6. The composition according to claim 1, wherein the vinegar syrup is taken 2-3 times a day in a quantity of 3-5 spoons.

7. The composition according to claim 1, wherein the parsomash is taken by thalassemia patients as tea and wherein the parsomash is a detoxifying agent.

8. The diet plan for thalassemia patient comprises:
a diet plan for anemia;
a diet plan for softening phlegm; and
a diet plan for excreting the phlegm.

9. The diet plan according to claim 8, wherein the diet plan for anemia comprises a currant, a fig, a prune, a jujube, a white basil seeds (dragon seeds), a sap taken from sheep backbone, a vinegar syrup, a passargad powder and a pars powder.

10. The diet plan according to claim 8, wherein the diet plan for softening the phlegm comprises a flexweld, a manna, and a plantain along with fumigation/vaporization.

11. The diet plan according to claim 8, wherein the diet plan for excreting the phlegm comprises a purgative and a snuffing material.

12. The diet plan according to claim 8 further comprises a breakfast, a lunch and a dinner.

13. The diet plan according to claim 12, wherein the breakfast comprises:
a drink comprising manna and flixweld;
a drink comprising white basil seeds and dragon head dissolved in rosewater and Bidmeshk;
pars powder;
grape sap; and
fruits comprising plantain, prune, fig and jujube.

14. The diet plan according to claim 12, wherein the lunch comprises:
a teaspoon or one or two tablets of passargad powder before lunch;
lunch meals comprising a pottage with beans, a green pea stew, sheep's rumen, abdomen and lung, cauliflower soup, cooked rice with coriander, sap taken from a sheep backbone, a grape sap, and a pars powder; and
a purgative material after lunch.

15. The diet plan according to claim 8 wherein the dinner comprises:
a teaspoon or one or two tablets of passargad powder before dinner;
a dinner meals comprising cooked rice with coriander or a boiled zucchini and fowl meat or a sheep's rumen, a sap taken from sheep backbone, grape sap, and pars powder.

16. The diet plan according to claim 8, further comprises snuffing, fumigation and oiling the nostrils before sleep.
